(19) United States
(12) Patent Application Publication
(10) Pub. No.: US 2008/0242967 A1
(43) Pub. Date: Oct. 2, 2008

(54) Medical imaging and therapy utilizing a scanned beam system operating at multiple wavelengths

(75) Inventor: Michael P. Weir, Blanchester, OH (US)

Correspondence Address: THOMPSON HINE L.L.P.;Intellectual Property Group, P.O. BOX 8801, DAYTON, OH, 45401-8801, US

(73) Assignee: Ethicon Endo-Surgery, Inc.

(21) Appl. No.: 11/728,944

(22) Filed: Mar. 27, 2007

(51) Int. Cl. A61B 5/00

(52) U.S. Cl. 600/407; 607/89

(57) ABSTRACT

An apparatus, for medically treating a patient, includes a scanned beam system including a radiation beam source assembly, a scanner, and a controller. The assembly is adapted to emit a radiation beam (such as, without limitation, a laser beam) at different wavelengths. The controller is operatively connected to the assembly and the scanner. The controller is adapted: to display an image of an area of the patient obtained by the scanned beam system; to receive from a user viewing the image an identification of an in-treatment region; and to control the assembly and the scanner to medically treat the in-treatment region using a therapeutic wavelength of the radiation beam which provides an increased scanned-beam-system-image contrast between the in-treatment region and an out-of-treatment region over another wavelength of the radiation beam available for therapy. A method for medically treating a patient using a scanned beam system is also disclosed.

### FIELD OF THE INVENTION

The present invention is related generally to scanned beam systems, and more particularly to a medical apparatus and to a method involving a scanned beam system.

### BACKGROUND OF THE INVENTION

Conventional scanned light beam systems, such as those adapted to function as bar code scanners, are available from Microvision, Inc. of Redmond, Wash.

An example of an endoscope application of a medical scanned laser beam imager is given in US Patent Application Publication 2005/0020926. The scanned laser beam imager includes a two-dimensional MEMS (micro-electromechanical system) scanner. The MEMS scanner is a dual-resonant-mirror scanner. The mirror scanner scans, about substantially orthogonal first and second axes, one or more light beams (such as light beams from red, green and blue lasers) through an optical dome at high speed in a pattern that covers an entire two-dimensional field of view or a selected region of a two-dimensional field of view. The scanned laser beam imager uses at least one light detector in creating a pixel image from the reflected light for display on a monitor.

It is known to medically treat a patient using a laser. Conventional medical lasers include a medical laser scalpel claimed in U.S. Pat. No. 4,421,382.

What is needed is an improved medical apparatus and method involving a scanned beam system.

### SUMMARY

An embodiment of the invention is for an apparatus for medically treating a patient. The apparatus includes a scanned beam system. The scanned beam system includes a radiation beam source assembly, a scanner, and a controller. The radiation beam source assembly is adapted to emit a radiation beam at different wavelengths. The controller is operatively connected to the radiation beam source assembly and the scanner. The controller is adapted: to display an image of an area of the patient obtained by the scanned beam system; to receive from a user viewing the image an identification of an in-treatment region; and to control the radiation beam source assembly and the scanner to medically treat the in-treatment region using a therapeutic wavelength of the radiation beam which provides an increased scanned-beam-system-image contrast between the in-treatment region and an out-of-treatment region over another wavelength of the radiation beam available for therapy. In one example, the therapeutic wavelength of the radiation beam substantially maximizes the contrast between the in-treatment region and the out-of-treatment region over any other wavelength of the radiation beam available for therapy.

A method of the invention is for medically treating a patient and includes steps a) through d). Step a) includes viewing an image of an area of the patient obtained by a scanned beam system. Step b) includes identifying an in-treatment region from viewing the image. Step c) includes selecting a therapeutic wavelength of the scanned beam system which provides an increased scanned-beam-system-image contrast between the in-treatment region and an out-of-treatment region over another wavelength of the radiation beam available for therapy. Step d) includes medically treating the in-treatment region with the scanned beam system using the therapeutic wavelength. In one example, step c) includes selecting a therapeutic wavelength of the scanned beam system which maximizes the contrast between the in-treatment region and the out-of-treatment region over any other wavelength of the radiation beam available for therapy.

Several benefits and advantages are obtained from the embodiment and/or method of the invention. In one example, by using a therapeutic wavelength of the radiation beam which substantially maximizes the scanned-beam-system-image contrast between the in-treatment region and an out-of-treatment region, medical treatment is provided to target sites (e.g., calculus sites and/or tissue sites) while lessening the medical effects of unavoidable treatment of non-target sites.

### BRIEF DESCRIPTION OF THE FIGURES

FIG. 1 is a schematic diagram of an embodiment of the invention including a scanned radiation beam system having a radiation beam source assembly, a scanner, at least one radiation detector, and a controller and including a side view of an internal portion of a patient;

FIG. 2 is taken along view 2-2 of FIG. 1 showing an in-treatment region and an out-of-treatment region within an area of the patient which corresponds to the field of view of the scanned beam system; and

FIG. 3 is a schematic diagram of an embodiment of the radiation beam source assembly of the scanned radiation beam system of FIG. 1.

### DETAILED DESCRIPTION

Before explaining an embodiment and method of the present invention in detail, it should be noted that each is not limited in its application or use to the details of construction and arrangement of parts and steps illustrated in the accompanying drawings and description. The illustrative embodiment and method of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiment and method of the present invention for the convenience of the reader and are not for the purpose of limiting the invention.

It is further understood that any one or more of the following-described enablements, applications, etc. can be combined with any one or more of the other following-described enablements, applications, etc.

U.S. patent application Ser. No. ______ , entitled MEDICAL DEVICE INCLUDING SCANNED BEAM UNIT FOR IMAGING AND THERAPY, and filed Mar. 12, 2007 [attorney docket no. END5764USNP], is incorporated by reference as if fully set forth herein.

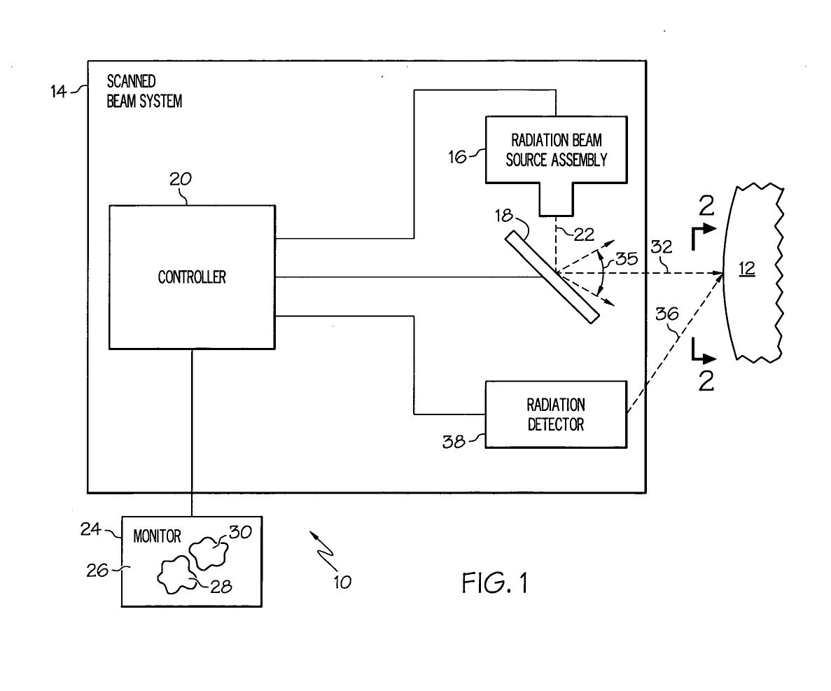

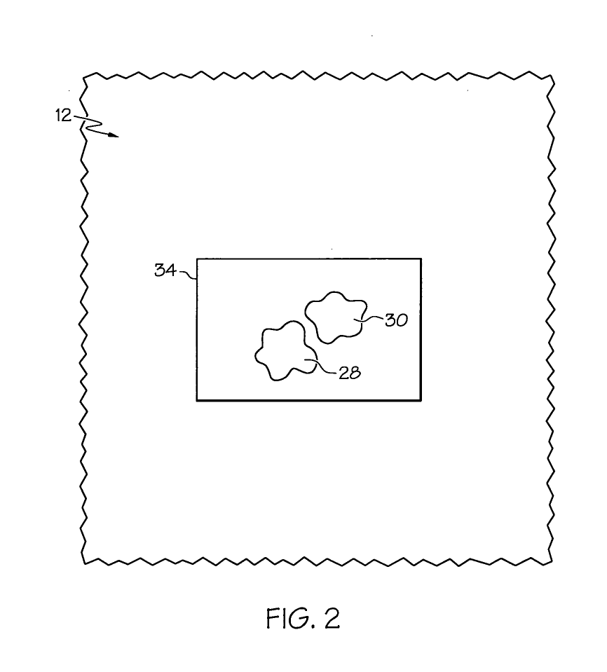

An embodiment of the invention is shown in FIGS. 1-3 and is for an apparatus 10 for medically treating a patient 12. The apparatus 10 includes a scanned beam system 14. The scanned beam system 14 includes a radiation beam source assembly 16, a scanner 18, and a controller 20. The radiation beam source assembly 16 is adapted to emit a radiation beam 22 at different wavelengths. The controller 20 is operatively connected to the radiation beam source assembly 16 and the scanner 18. The controller 20 is adapted: to display an image 26 of an area 34 of the patient 12 obtained by the scanned beam system 14; to receive from a user viewing the image 26 an identification of an in-treatment region 28; and to control the radiation beam source assembly 16 and the scanner 18 to medically treat the in-treatment region 28 using a therapeutic wavelength of the radiation beam 22 which provides an increased scanned-beam-system-image contrast between the in-treatment region 28 and an out-of-treatment region 30 over another wavelength of the radiation beam 22 available for therapy.

It is noted that “therapy” means treatment of a medical condition. It is also noted that the controller 20 is adapted to display the image 26 whether or not the radiation beam 22 is a light beam. Examples of light beams include laser light beams and non-laser light beams. Examples of radiation beams, other than light beams, are left to those skilled in the art. It is further noted that the components of the scanned beam system 14 may or may not be disposed within a single housing. In one employment of the embodiment of FIGS. 1-3, the apparatus 10 includes a monitor 24, and the controller 20 is adapted to display the image 26 on the monitor 24.

In one example of the embodiment, the therapeutic wavelength of the radiation beam 22 substantially maximizes the contrast between the in-treatment region 28 and the out-of-treatment region 30 over any other wavelength of the radiation beam 22 available for therapy. In one variation, the controller 20 is adapted to determine the maximum contrast based on a survey of the area 34 with the scanned beam system 14 using the radiation beam source assembly 16 at different wavelengths. In one illustration, contrast between two regions is determined from the difference between the detected radiation beam signal strengths from the two regions divided by the sum of the detected radiation beam signal strengths from the two regions wherein a detected signal strength from a region is an average of the detected signal strengths over the region. Other definitions of contrast are left to the artisan.

In one enablement of the embodiment, the controller 20 is adapted to receive a therapeutic wavelength selection from the user based on the user viewing images of the area 34 obtained by the scanned beam system 14 at different wavelengths. In a first variation, the user chooses a therapeutic wavelength which substantially maximizes the contrast between the in-treatment region 28 and the out-of treatment region 30 for all wavelengths available for therapy. In one modification, the user employs a controller-connected marking pen (not shown) on a monitor 24 displaying the image 26 to identify to the controller 20 the in-treatment region 28 and to optionally identify to the controller 20 the out-of-treatment region 30. In a second variation, the user considers other factors, in addition to contrast, such as predetermined medical effectiveness of a wavelength availably for therapy, reflections, penetration, etc.

In one application of the embodiment, the in-treatment region 28 is a calculus (such as, but not limited to, a kidney stone) of the patient 12. In another application, the in-treatment region 28 is tissue of the patient 12. Other applications are left to those skilled in the art.

In one arrangement of the embodiment, the radiation beam 22 from the radiation beam source assembly 16 is received by the scanner 18 and reflected as a scanned radiation beam 32 which strikes the patient 12 within an area 34 corresponding to a field of view 35 of the scanned beam system 14. Reflected radiation 36 from the patient 12 is received by a radiation detector 38 and sent as a signal to the controller 20. As used in the present application, “reflected radiation 36” means radiation which has been detected by the radiation detector 38 whether from true reflection, scattering, and/or refraction, etc. It is noted that the unlabeled solid lines between components in FIG. 1 represent connections between the components.

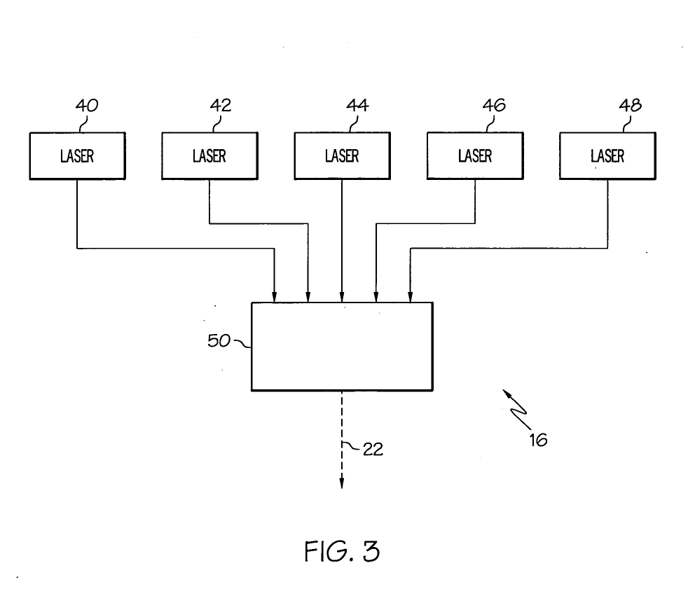

In the same or a different arrangement of the embodiment, the radiation beam source assembly 16, as shown in FIG. 3, includes a plurality of radiation beam sources 40, 42, 44, 46 and 48 which may comprise, for example, three low power imaging radiation beam sources which may or may not have high power therapy capability and two high power therapy radiation beam sources whose wavelengths are tunable or fixed. Other arrangements and examples are left to the artisan. It is noted that the unlabeled solid lines having directional arrowheads in FIG. 3 represent radiation from the radiation beam sources 40, 42, 44, 46 and 48 before such radiation leaves the radiation beam source assembly 16. In one example, the radiation beam source assembly 16 includes a combiner 50 controlled by the controller 20 and adapted to either pass radiation from only one of the radiation beam sources 40, 42, 44, 46 and 48 or to combine radiation from two or more radiation beam sources 40, 42, 44, 46 and 48. In the same or a different example, the radiation beam sources 40, 42, 44, 46 and 48 are lasers (i.e., the radiation beam source assembly 16 is a laser beam source assembly).

A method of the invention is for medically treating a patient and includes steps a) through d). Step a) includes viewing an image 26 of an area 34 of the patient 12 obtained by a scanned beam system 14. Step b) includes identifying an in-treatment region 28 from viewing the image 26. Step c) includes selecting a therapeutic wavelength of the scanned beam system 14 which provides an increased scanned-beam-system-image contrast between the in-treatment region 28 and an out-of-treatment region 30 over another wavelength of the radiation beam 22 available for therapy. Step d) includes medically treating the in-treatment region 28 with the scanned beam system 14 using the therapeutic wavelength. It is noted that any one or more or all of steps a), b) and c) may be performed by a user or may be performed by a machine (wherein the machine may or may not be part of the scanned beam system 14). In one illustration, step a) includes viewing the image 26 on a monitor 24.

In one employment of the method, step b) includes identifying the in-treatment region 28 from viewing the image 26 which provides the greatest contrast between the in-treatment region 28 and the out-of-treatment region 30 from a plurality of viewed images obtained at different imaging wavelengths and combinations thereof. In one variation, the user then marks the in-treatment region 28 by outlining it on the image 26 displayed on a monitor 24 using a marking pen (not shown) which is operatively connected to a controller 20 of the scanned beam system 14.

In the same or a different employment of the method, step c) includes selecting a therapeutic wavelength of the scanned beam system 14 which substantially maximizes the contrast between the in-treatment region 28 and the out-of-treatment region 30 over any other wavelength of the radiation beam 22 available for therapy. In one variation, a controller 20 of the scanned beam system 14 determines the maximum contrast.

In one enablement of the method, a user selects the therapeutic wavelength based on viewing images of the area 34 obtained by the scanned beam system 14 at different wavelengths.

In one application of the method, the in-treatment region 28 is a calculus of the patient 12. In another application, the in-treatment region 28 is tissue of the patient 12.

While the present invention has been illustrated by a description of an expression of an embodiment and method, it is not the intention of the applicant to restrict or limit the spirit and scope of the appended claims to such detail. Numerous other variations, changes, and substitutions will occur to those skilled in the art without departing from the scope of the invention. It will be understood that the foregoing description is provided by way of example, and that other modifications may occur to those skilled in the art without departing from the scope and spirit of the appended Claims.

What is claimed is:

1. An apparatus for medically treating a patient comprising a scanned beam system, wherein the scanned beam system includes a radiation beam source assembly, a scanner, and a controller, wherein the radiation beam source assembly is adapted to emit a radiation beam at different wavelengths, wherein the controller is operatively connected to the radiation beam source assembly and the scanner, and wherein the controller is adapted:
to display an image of an area of the patient obtained by the scanned beam system;
to receive from a user viewing the image an identification of an in-treatment region; and
to control the radiation beam source assembly and the scanner to medically treat the in-treatment region using a therapeutic wavelength of the radiation beam which provides an increased scanned-beam-system-image contrast between the in-treatment region and an out-of-treatment region over another wavelength of the radiation beam available for therapy.

2. The apparatus of claim 1, wherein the therapeutic wavelength of the radiation beam substantially maximizes the contrast between the in-treatment region and the out-of-treatment region over any other wavelength of the radiation beam available for therapy.

3. The apparatus of claim 2, wherein the controller is adapted to determine the maximum contrast based on a survey of the area with the scanned beam system using the radiation beam source assembly at different wavelengths.

4. The apparatus of claim 1, wherein the controller is adapted to receive a therapeutic wavelength selection from the user based on the user viewing images of the area obtained by the scanned beam system at different wavelengths.

5. The apparatus of claim 1, wherein the in-treatment region is a calculus of the patient.

6. The apparatus of claim 1, wherein the in-treatment region is tissue of the patient.

7. A method for medically treating a patient comprising the following steps:
a) viewing an image of an area of the patient obtained by a scanned beam system;
b) identifying an in-treatment region from viewing the image;
c) selecting a therapeutic wavelength of the scanned beam system which provides an increased scanned-beam-system-image contrast between the in-treatment region and an out-of-treatment region over another wavelength of the radiation beam available for therapy; and
d) medically treating the in-treatment region with the scanned beam system using the therapeutic wavelength.

8. The method of claim 7, wherein step b) includes identifying the in-treatment region from viewing the image which provides the greatest contrast between the in-treatment region and the out-of-treatment region from a plurality of viewed images obtained at different imaging wavelengths and combinations thereof.

9. The method of claim 7, wherein step c) includes selecting a therapeutic wavelength of the scanned beam system which substantially maximizes the contrast between the in-treatment region and the out-of-treatment region over any other wavelength of the radiation beam available for therapy.

10. The method of claim 9, wherein a controller of the scanned beam system determines the maximum contrast.

11. The method of claim 7, wherein a user selects the therapeutic wavelength based on viewing images of the area obtained by the scanned beam system at different wavelengths.

12. The method of claim 7, wherein the in-treatment region is a calculus of the patient.

13. The method of claim 7, wherein the in-treatment region is tissue of the patient.
